(19) United States
(12) Patent Application Publication
(10) Pub. No.: US 2010/0069876 A1
(43) Pub. Date: Mar. 18, 2010

(54) Baby Napkin Aid

(76) Inventor: Melanie Louise Sonter, Coolum Beach (AU)

Correspondence Address: SQUIRE, SANDERS & DEMPSEY L.L.P., 1 MARITIME PLAZA, SUITE 300, SAN FRANCISCO, CA, 94111, US

(21) Appl. No.: 12/538,753

(22) Filed: Aug. 10, 2009

(30) Foreign Application Priority Data
Aug. 11, 2008 (AU) 2008203797

(51) Int. Cl. A61F 13/15

(52) U.S. Cl. 604/393

(57) ABSTRACT

An aid (1) is described for use with a disposable napkin (11) of the type worn by a baby (10) for incontinence and fastened in place with one or more re-usable pressure-sensitive fixing tabs 12(a,b). The baby napkin aid (1) comprises a shaped flexible panel (2) manufactured from two layers 2(a,b) of flexible material. The lower portion of the panel (2) is bifurcated into two arms 3(a,b). A first button (4a) is sewn onto the upper portion of the panel (2) near the side edge (5a). A second button (4b) is sewn onto the upper portion of the panel (2) near the opposite side edge (5b). Button holes 6(a,b), to accommodate the respective buttons 4(a,b) are placed in the respective arms 3(a,b) of the panel (2). A flexible strap (7) is affixed at one end to the side edge (5a) at the upper portion of the panel (2). Sandwiched between the two layers 7(a,b) is an elasticized tape (8). At the other end of the strap (7) are two button holes 8(a,b), each being able to be selected for connection to the button (4a) as required.

THIS INVENTION relates to a baby napkin. In particular, it is directed to an aid for use with a disposable baby napkin to prevent the removal of such a napkin worn by a baby.

Although specific references will be made to a baby napkin, it will be understood that these are for illustrative purposes only, are not limiting and that this invention could be used in other applications.

Further, throughout this specification, unless the contrary is expressly stated, where a document, act or item of knowledge is referred to or discussed, this to reference or discussion is not an admission that the document, act or item of knowledge or any combination thereof was, at the priority date, publicly available, known to the public, part of the common general knowledge, or known to be relevant to an attempt to solve any problem with which this specification is concerned.

Two types of baby napkin are currently in common use. The first is the traditional square cloth napkin which is held in place by one or more safety pins. Some parents and other carers are concerned at the use of these sharp pins in close proximity to a baby, others find it too time-consuming to configure and fit the napkin, and yet others simply find it difficult to shape and fit such a napkin. Therefore, a second type of baby napkin—the disposable type—has gained increasing popularity. Disposable baby napkins are generally pre-shaped to easily position around the buttocks of a baby, and they do not require the use of safety pins as they are held in place by one or more overlapping pressure-sensitive tabs which are held together either by hook and loop members or are coated with a re-usable adhesive.

However, one disadvantage of these attachment mechanisms for disposable napkins is that they can readily be disengaged by the baby, allowing the napkin to fall off, unnoticed by a parent or carer, with its obvious attendant problems. Further, continuously disengaging and re-engaging these tabs weakens the effectiveness of the attachment and the napkin may have to be disposed of earlier than necessary simply because it can no longer be held in position.

It is thus a general object of the present invention to overcome, or at least ameliorate, one or more of the above-mentioned disadvantages.

The present inventor has developed an aid for use in conjunction with the traditional disposable baby napkin which covers the afore-mentioned pressure-sensitive fixing tabs preventing access thereto by the baby.

Therefore, according to a first aspect of the present invention, there is provided an aid for use with a napkin of the type worn by a baby for incontinence and fastened in place with one or more re-usable pressure-sensitive fixing tabs, said aid including:
a flexible panel defined by an inside surface for facing toward said infant, an outside surface facing away from said infant, an upper portion close to the waist region of said infant, and a lower portion being bifurcated to define a first arm and a second arm;
a first securing means having a first component close to a first side edge of said panel within said upper portion, and a second component associated with said first arm and reversibly operatively connectable to said first component;
a second securing means having a first component close to an opposing second side edge of said panel within said upper portion, and a second component associated with said second arm and reversibly operatively connectable to said first component; and
a strap fixed to said first side edge within said upper portion adapted to extend around the rear waist region to the front waist region of said infant for reversible operable connection to said second securing means.

Each of said first and second securing means may be independently selected from the group comprising a button and button hole combination, press studs & other snap-like fasteners or zip fasteners.

In those embodiments wherein said first and/or second securing means is a button and button hole combination, said button may be attached to said inside surface close to a respective side edge of said panel within said upper portion, and said button hole may be in the respective said first or second arm.

In those embodiments wherein said first and/or second securing means is a press stud or similar snap fastener, the male interlocking disc may be attached to said inside surface close to a respective side edge of said panel within said upper portion, and the female interlocking disc is in the respective said first or second arm.

In those embodiments wherein said first and/or second securing means is a zip fastener, the first strip of toothed tape may be attached to said inside surface close to a respective side edge of said panel within said upper portion, and the second strip of toothed tape is in the respective said first or second arm.

Said strap may be adjustable in length.

When said strap is adjustable in length, in one embodiment, the required adjustability may be obtained by use of an elasticized strap; in another embodiment, said strap may include a selection of multiple spaced connectors to said second securing means.

Preferably, said flexible panel has said inside and outside surfaces manufactured from a fabric material,

Said flexible panel may be multi-layered.

When said flexible panel is multi-layed, an internal layer may be manufactured from a liquid impervious material.

As a second aspect of the present invention, there is provided a method to restrict access by a baby to any re-usable pressure-sensitive fixing tab on a disposable-type napkin of the type worn by said baby for incontinence, said method including:
fitting said napkin to said baby;
obtaining an aid as herein before described;
placing said inside surface of said aid on the front waist region of said infant;
positioning said first arm around one leg of said infant and securing said first securing means;
positioning said second arm around the other leg of said infant and securing said second securing means;
extending said strap around the rear waist region to the front waist region of said infant; and
securing said strap to said second securing means ensuring that said upper portion of said panel covers said pressure-sensitive fixing tab.

A preferred embodiment of the present invention will now be described with reference to the accompanying drawings, wherein:

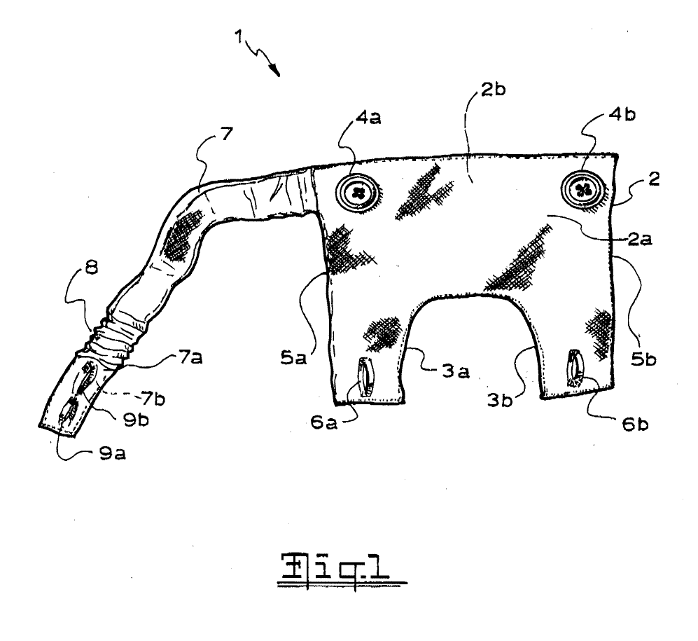

FIG. 1 is a plan view of an aid constructed in accordance with the present invention;

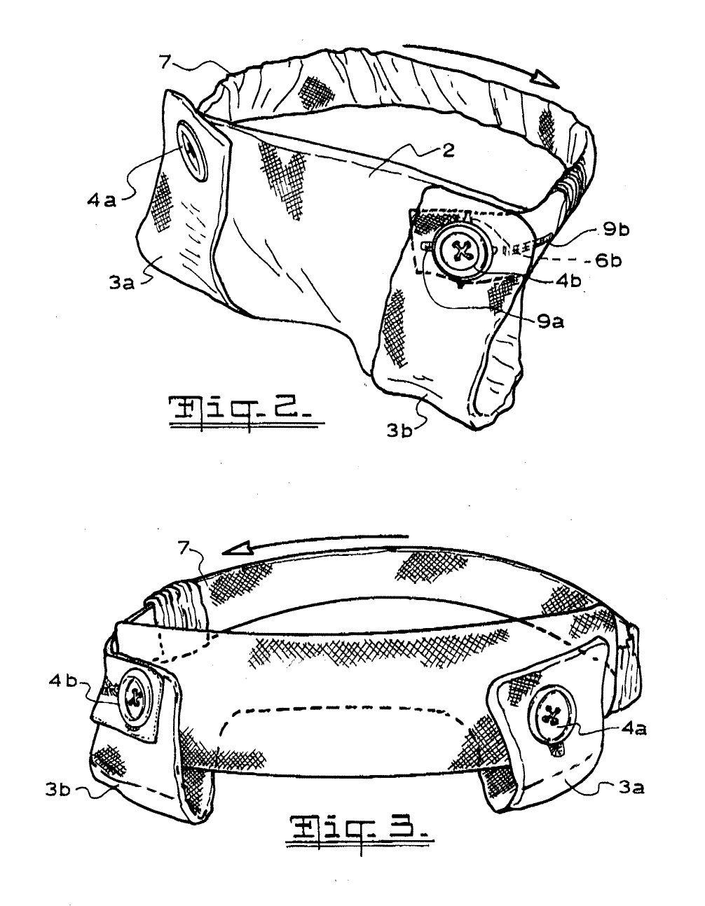

FIG. 2 is a sketch of a first configuration of the aid of FIG. 1;

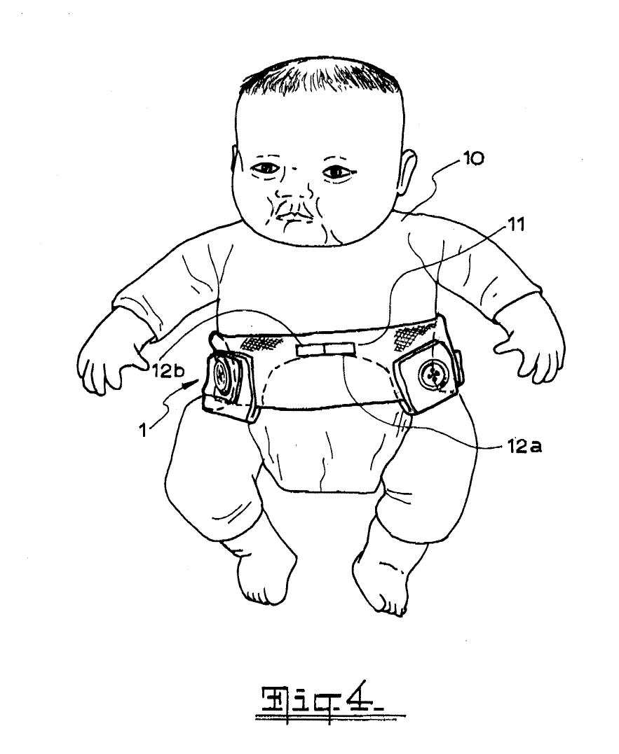

FIG. 3 is a sketch of a second configuration of the aid of FIG. 1; and

FIG. 4 is the aid of FIG. 3 in use.

Referring to FIG. 1, the baby napkin aid (1) comprises a shaped flexible panel (2) manufactured from two layers 2(a,b) of any suitable flexible material sewn together. The lower portion of the panel (2) is bifurcated into two arms 3(a,b). A first button (4a) is sewn onto the upper portion of the panel (2) near the side edge (5a). A second button (4b) is sewn onto the upper portion of the panel (2) near the opposite side edge (5b). Button holes 6(a,b), to accommodate the respective buttons 4(a,b) are placed in the respective arms 3(a,b) of the panel (2). A flexible strap (7), manufactured from two layers 7(a,b) of any suitable material sewn together but, preferably, being of the same material as the layers 2(a,b) of the panel (2), is affixed at one end to the side edge (5a) at the upper portion of the panel (2). Sandwiched between the two layers 7(a,b) is an elasticized tape (8) of the type well known in the art to allow the strap (7) to be reversibly extended. At the other end of the strap (7) are two button holes 8(a,b), each being able to be selected for connection to the button (4a) as required when the aid (1) is in use.

In use, in one configuration as depicted in FIG. 2, the surface (2b) of the panel (2) is placed on the front waist region of a baby (not illustrated) wearing a disposable napkin (not illustrated) so that the upper portion of the panel is covering the pressure-sensitive tab(s) that hold the napkin in place. The arm (3a) is wrapped around one leg of the baby and the button (4a) is secured through the button hole (6a). The arm (3b) is wrapped around the other leg of the baby and the button (4b) is secured through the button hole (6b). The strap (7) is then extended around the rear waist region of the baby to be secured at the front waist region by button (4b) through button hole (9a) or, if a tighter fit is required, through button hole (9b). The elasticized nature of the strap (7) allows for varying waist diameters of babies and also assists in is tensioning the aid (1) in place.

Should less coverage of the waist region be required for any particular baby, the aid (1) can be fixed in a second configuration as depicted in FIG. 3. In this configuration, the surface (2a) of the panel (2) is placed on the front waist region of a baby (not illustrated) wearing a disposable napkin (not illustrated). A section of the upper portion of the panel (2) is then folded forwards, ensuring that the remainder of the upper portion of the panel (2) is covering the pressure-sensitive tab(s) that hold the napkin in place. The arm (3a) is wrapped around one leg of the baby and the button (4a) is secured through the button hole (6a). The arm (3b) is wrapped around the other leg of the baby and the button (4b) is secured through the button hole (6b). The strap (7) is then extended around the rear waist region of the baby to be secured at the front waist region by button (4b) through button hole (9a) or, if a tighter fit is required, through button hole (9b). The elasticized nature of the strap (7) still allows for varying waist diameters of babies and also assists in tensioning the aid (1) in place.

FIG. 4 depicts a baby (10) wearing a disposable napkin (11) held in place by pressure-sensitive tabs 12(a,b). The aid (1), in the configuration described above with reference to FIG. 3, is placed in position over the napkin (11), the upper portion of the panel (2) thus covering the tabs 12(a,b).

The present invention thus provides a number of advantages which include:
parents and carers can still use the convenient disposable napkins without the worry of the baby tampering with the tabs thus possibly removing the napkin unseen;
any given example of the aid can be configured in two ways to provide a range of coverage of the waist region of a baby, thus reducing the number of sizes of the aid likely to be required as a baby grows;
the aid can be manufactured from readily available materials which can be readily laundered; and
provides a surface upon which designs, promotional material and the like can be placed if required.

It will be appreciated that the above described embodiment is only an exemplification of the various aspects of the present invention and that modifications and alterations can be made thereto without departing from the inventive concept as defined in the following claims.

1. An aid for use with a napkin of the type worn by a baby for incontinence and fastened in place with one or more re-usable pressure-sensitive fixing tabs, said aid including:
a flexible panel defined by an inside surface for facing toward said infant, an outside surface facing away from said infant, an upper portion close to the waist region of said infant, and a lower portion being bifurcated to define a first arm and a second arm;
a first securing means having a first component close to a first side edge of said panel within said upper portion, and a second component associated with said first arm and reversibly operatively connectable to said first component;
a second securing means having a first component close to an opposing second side edge of said panel within said upper portion, and a second component associated with said second arm and reversibly operatively connectable to said first component; and
a strap fixed to said first side edge within said upper portion adapted to extend around the rear waist region to the front waist region of said infant for reversible operable connection to said second securing means.

2. An aid as defined in claim 1 wherein, each of said first and/or second securing means is independently selected from the group comprising a button and button hole combination, press studs & other snap-like fasteners, or zip fasteners.

3. An aid as defined in claim 2 wherein, each said first and/or second securing means is a button and button hole combination.

4. An aid as defined in claim 3 wherein, said button is attached to said inside surface close to a respective side edge of said panel within said upper portion, and said button hole is in the respective said first or second arm.

5. An aid as defined in claim 1 wherein, said strap is adjustable in length.

6. An aid as defined in claim 5 wherein, said strap is an elasticized strap.

7. An aid as defined in claim 1 wherein, said strap includes a selection of multiple connectors to said second securing means.

8. An aid as defined in claim 1 wherein, said flexible panel has said inside and outside surfaces manufactured from a fabric material.

9. An aid as defined in claim 1 wherein, said flexible panel is multi-layered.

10. An aid as defined in claim 9 wherein, an internal layer is manufactured from a liquid impervious material.

11. A method to restrict access by a baby to any re-usable pressure-sensitive fixing tab on a disposable-type napkin of the type worn by said baby for incontinence, said method including:
fitting said napkin to said baby;
obtaining an aid as defined in claim 1;
placing said inside surface of said aid on the front waist region of said infant;
positioning said first arm around one leg of said infant and securing said first securing means;
positioning said second arm around the other leg of said infant and securing said second securing means;
extending said strap around the rear waist region to the front waist region of said infant; and
securing said strap to said second securing means ensuring that said upper portion of said panel covers any said pressure-sensitive fixing tab.
